(19) United States
(12) Patent Application Publication
(10) Pub. No.: US 2026/0144574 A1
(43) Pub. Date: May 28, 2026

(54) MODULAR TULIP INSERTER

(71) Applicant: Astura Medical Inc., Iriving, TX (US)

(72) Inventors: Keith klausman, Irving, TX (US); Thomas Purcell, Irving, TX (US)

(73) Assignee: Astura Medical Inc., Irving, TX (US)

(21) Appl. No.: 18/961,383

(22) Filed: Nov. 26, 2024

(51) Int. Cl. A61B 17/70; A61B 17/86

(52) U.S. Cl. CPC A61B 17/7037; A61B 17/7032; A61B 17/8685

(57) ABSTRACT

A modular tulip inserter is disclosed for use in attaching a modular tulip assembly to a screw shank, the modular tulip inserter includes a distal end configured to hold modular tulip assembly and a proximal end having a handle and a lever that is actuated to attach the modular tulip assembly to the screwhead.

### CROSS-REFERENCE TO RELATED APPLICATIONS

N/A.

### FIELD

The present invention relates generally to the field of spinal fusion surgery, and more specifically, to a modular tulip inserter used to attach a modular tulip assembly to a screw shank during surgery.

### BACKGROUND

Certain spinal conditions, including a fracture of a vertebra and a herniated disc, indicate treatment by spinal immobilization. Several methods of spinal joint immobilization are known, including surgical fusion and the attachment of pins and bone plates to the affected vertebras. One known device is a stabilization rod interconnecting the two or more pedicle screws to stabilize the vertebras spanned by the screws. The screw includes a channel for receiving the stabilization rod and a nut to apply compressive force between the rod and the screwhead to firmly fix the rod between the spanned vertebras and thus stabilize the spinal vertebrae.

One problem with utilizing conventional pedicle screws is that the tulip can be large and cause limited visibility when using a minimally invasive approach and performing disc preparation.

Another problem is the number of different screws sizes for implant/screw selection in larger cases where a surgeon is implanting 10 plus screws.

Another problem is the high cost and inventory for all the different sizes that are required.

It may be desirable to provide an instrument to install modular tulips on an implanted screw shank that solves the problems above.

### SUMMARY

The present invention is directed to a modular tulip inserter that is used to attach a modular tulip to a screw shank during a procedure. Attaching the modular tulip with the modular tulip inserter during the procedure allows maximum visibility of the site when using a minimally invasive approach and performing disc preparation.

The modular tulip inserter allows the surgeon to first implant the screw shanks, then select a desired modular tulip from various modular tulips, then attach the desired modular tulip with the modular tulip inserter.

By utilizing a modular tulip inserter to attach a modular tulip, a set now does not need a tulip for every screw shank, there only needs to be enough tulip options to cover the case.

According to some embodiments, a modular tulip inserter designed to attach to a modular tulip assembly comprises: tulip engagement clips having a distal end configured to attach to a modular tulip body; an actuation lever on a proximal end; and an internal shaft having a proximal end connected to the actuation lever and a distal end configured to contact a modular tulip bushing positioned within the modular tulip body; wherein a pull action of the actuation lever translates the internal shaft distally to apply a downward force to the modular tulip bushing to translate the modular tulip bushing distally in the modular tulip body to a screwhead captured state.

According to some embodiments, a modular tulip inserter designed to attach to a modular tulip assembly comprises: a handle on the proximal end configured to provide grip for a surgeon; tulip engagement clips having a distal end configured to attach to a modular tulip body; an actuation lever connected to an internal shaft via a linkage which converts a pull action of the actuation lever to a linear translation of the internal shaft, and a distal end of the internal shaft being configured to contact a modular tulip bushing positioned within the modular tulip body; wherein the pull action of the actuation lever translates the internal shaft distally to apply a downward force to the modular tulip bushing to translate the modular tulip bushing distally in the modular tulip body to a screwhead captured state; and a force limiting spring coupled to the internal shaft designed to limit the amount of downward force applied to the modular tulip bushing; and a lever return spring coupled to the internal shaft configured to: compress when the actuation lever is pulled, and expand when the actuation lever is released to return the actuation lever to the starting position.

According to some embodiments, a modular tulip inserter designed to attach to a modular tulip assembly comprises: a handle on the proximal end configured to provide grip for a surgeon; tulip engagement spring-loaded clips configured to separate when inserted on a modular tulip body until the distal end is coupled to the modular tulip body to lock the modular tulip assembly on the distal end; an actuation lever connected to an internal shaft via a linkage which converts a pull action of the actuation lever to a linear translation of the internal shaft, and a distal end of the internal shaft being configured to contact a modular tulip bushing positioned within the modular tulip body; wherein the pull action of the actuation lever translates the internal shaft distally to apply a downward force to the modular tulip bushing to translate the modular tulip bushing distally in the modular tulip body to a screwhead captured state; and a force limiting spring coupled to the internal shaft designed to limit the amount of downward force applied to the modular tulip bushing; and a lever return spring coupled to the internal shaft configured to: compress when the actuation lever is pulled, expand when the actuation lever is released to return the actuation lever to the starting position; and an indicator button having a visual mark designed to provide visual indication when the internal shaft translates distally a full distance.

### BRIEF DESCRIPTION OF THE DRAWINGS

FIG. 1 is a side view showing one embodiment of a modular tulip inserter designed to deliver a modular tulip assembly to an implanted screw.

FIG. 2 is a sectional side view of FIG. 1.

FIG. 3 is a perspective exploded view showing components of the modular tulip assembly.

FIG. 4 is a side view showing the modular tulip bushing positioned within U-shaped tulip body.

FIG. 5 is a side view showing modular tulip assembly being lowered on a pedicle screw that has been implated.

FIG. 6 is a side view of the modular tulip assembly in a locked state on the pedicle screw.

FIGS. 7A-7D show an overview of attachment and delivery of a modular tulip assembly to a pedicle screw using a modular tulip inserter.

FIGS. 8A-8D are sectional views showing more details of a modular tulip inserter delivering a modular tulip assembly to a pedicle screw.

FIGS. 9A-9D are sectional views showing details of a modular tulip inserter with an attached modular tulip assembly for delivery to a screwhead.

FIGS. 10A-10D show an indicator button with a visual mark positioned in the inline coupling designed to provide visual indication that the modular tulip assembly has been set into the captured state on a screwhead.

### DETAILED DESCRIPTION

A modular tulip inserter is disclosed for use in attaching a modular tulip to a screw shank.

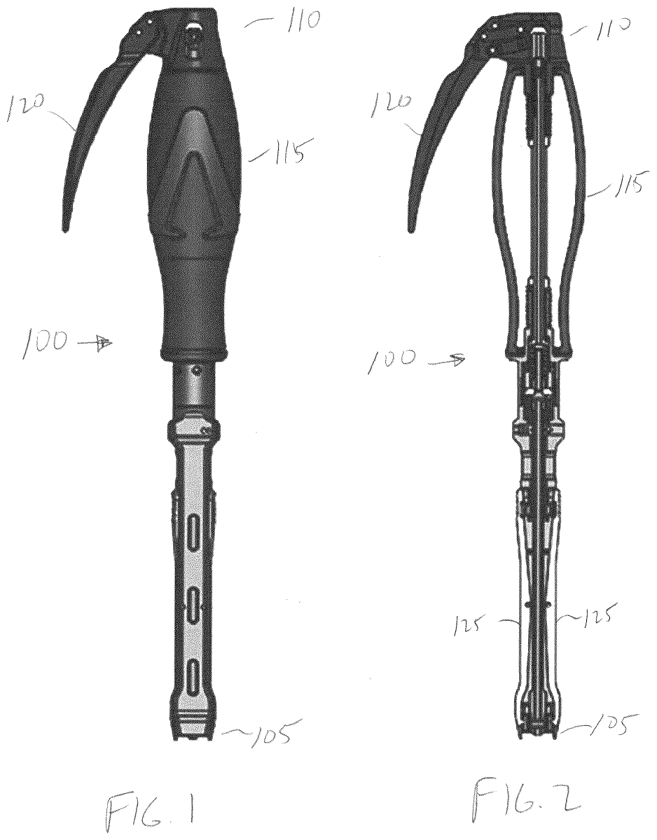

FIG. 1 is a side view and FIG. 2 is a sectional side view showing one embodiment of a modular tulip inserter 100 designed to deliver a modular tulip assembly 200 to an implanted screw. The modular tulip inserter 100 includes a distal end 105 configured to hold modular tulip assembly 200 and a proximal end 110 having a handle 115 and an actuation lever 120. The actuation lever 120 being configured to lock the modular tulip assembly 200 to the screw.

FIG. 3 is a perspective exploded view showing components of the modular tulip assembly 200 including a-shaped tulip body 205 and a modular tulip bushing 210. The modular tulip bushing 210 includes flexible members 212 around a lower opening configured to receive a screwhead, and a tapered distal external profile for locking against the inside of the U-shaped tulip body 205 and squeezing the screwhead 315 with the flexible members 212. The modular tulip bushing 210 also includes two stage cut outs on both sides, the first stage cut out is a lower cut out 235 and the second stage cut out is an upper cut out 240. The two stage cut outs interact with flexible wires 215 to lock the modular tulip bushing 210 within the U-shaped tulip body 205 and prevent backout. The first stage cut outs position the modular tulip assembly 200 in an unlocked state with the lower cut outs 235 locked with the flexible wires 215. The second stage cut outs position the modular tulip assembly 200 in a locked state with the upper cut outs 240 locked with the flexible wires 215.

During assembly, the modular tulip bushing 210 is inserted into a central opening of the U-shaped tulip body 205. The modular tulip bushing 210 is configured to translate downward inside the U-shaped tulip body 205 to contact and flex or compress the flexible wires 215 into radial grooves in the wall of the U-shaped tulip body 205. In the first stage, the flexible wires 215 spring back straight when they engage the lower cut outs 235 and prevent upward translation or backing out of the modular tulip bushing 210 from the U-shaped tulip body 205. The lower cut outs 235 include a lower portion that is shaped to engage the flexible wire 215 to prevent upward translation of the modular tulip bushing 210, and an upper ramped or tapered portion that is shaped to engage and flex or compress the flexible wire 215 into the grooved slot 245 to allow continued downward translation of the modular tulip bushing 210 from the first stage to the second stage. In the first stage, the modular tulip assembly 200 is in an unlocked state within modular tulip bushing 210 configured to be pushed on a screwhead.

The second stage upper cut out 240 engages the flexible wire 215 that has been compressed by the ramped or tapered portion in stage one as the modular tulip bushing 210 continues to translate downward in the U-shaped tulip body 205. The flexible wires 215 spring back straight when they engage the upper cut outs 240. The upper cut outs 240 include a lower portion that is shaped to engage the flexible wire 215 to prevent upward translation of the modular tulip bushing 210. In the second stage, the modular tulip bushing 210 is in a locked state within the U-shaped tulip body 205 to secure and lock the screwhead in the modular tulip assembly 200.

Flexible wires 215 may provide an audible sound and tactile feedback when transitioning the modular tulip bushing 210 from the unlocked state to the locked state. This feedback gives the surgeon confidence that the change from the unlocked state to the locked state was completed successfully. The flexible wire 215 also stops the modular tulip bushing 210 from upward translation back into the unlocked state, which eliminates the possibility of the modular tulip assembly 200 disengaging the screwhead 315 accidentally. The addition of flexible wires 215 also requires a removal tool to bring the modular tulip bushing 210 back to the unlocked state for removal.

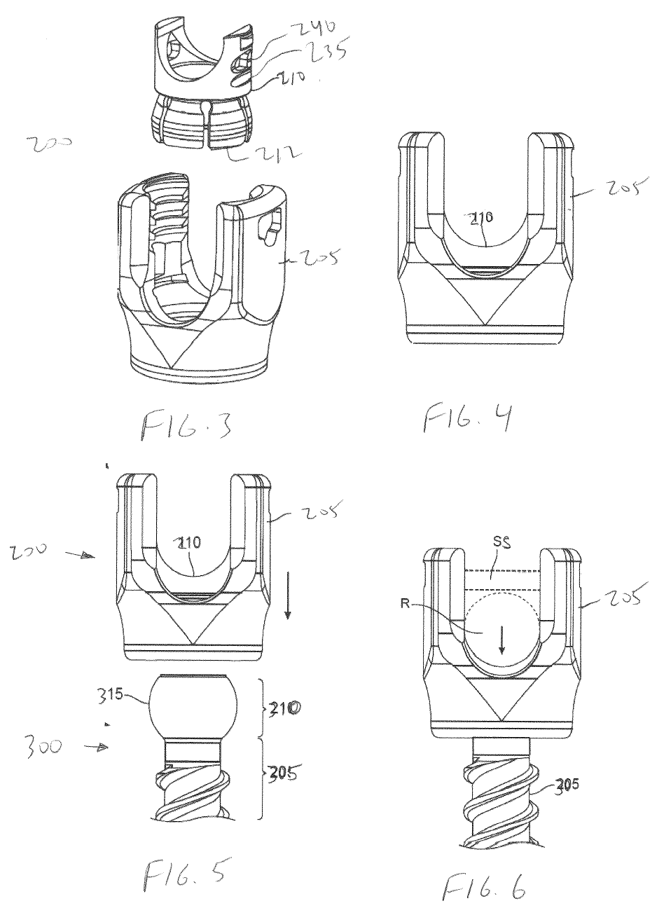

FIG. 4 is a side view showing the modular tulip bushing 210 positioned within U-shaped tulip body 205. The U-shaped tulip body 205 and modular tulip bushing 210 are configured to hold a rod R. The figure shows the modular tulip assembly 200 in the delivery state or unlocked state, with the modular tulip bushing 210 unlocked within the tulip body 205.

FIG. 5 is a side view showing modular tulip assembly 200 being lowered on a pedicle screw 300 that has been implated. Attaching the modular tulip assembly 200 later in the surgery allows maximum visibility when using a minimally invasive approach. The pedicle screw 300 includes a distal end having a screw shank 305 and a proximal end having a screwhead 310 with a curved outer surface 315. Once the screw shank 305 has been implanted, the surgeon selects a modular tulip assembly 200 from the various modular tulip assembly options. The modular tulip assembly 200 is positioned on the screwhead 210 in the unlocked state, with the modular tulip bushing 210 being in the unlocked position within the tulip body 205.

FIG. 6 is a side view of the modular tulip assembly 200 in a locked state on the pedicle screw 300. The interior of the U-shaped tulip body 205 includes a threaded portion to receive a rod locking device, such as a set screw SS. The rod R is positioned in the modular tulip assembly 200, and the set screw is configured to reduce the rod R into the U-shaped tulip body 205 and push the rod R 400 against the lower portion to lock the rod in the modular tulip assembly 200.

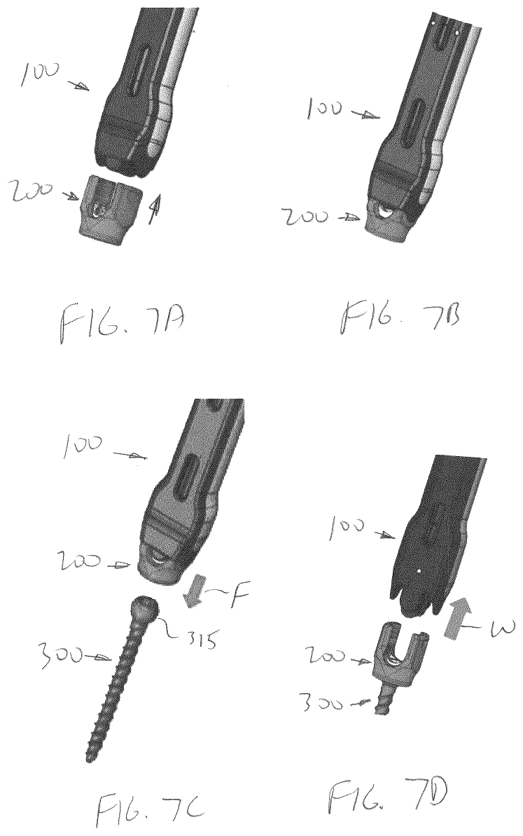

FIGS. 7A-7D show an overview of a modular tulip inserter 100 used for attachment and delivery of a modular tulip assembly 200 to a pedicle screw 300. The modular tulip assembly 200 attaches to the modular tulip inserter 100 by way of spring-loaded clips holding it in place. The modular tulip inserter 100 is configured to exert a downward force F to snap the modular tulip assembly 200 onto the screwhead 315 of the pedicle screw 300. An audible snap and tactile feedback indicate the modular tulip assembly 200 has completely engaged the screwhead 310 of the screw 300. Next, the surgeon pulls the lever 120 to lock the modular tulip bushing 210 in a captured state within the U-shaped tulip body 205. The captured state prevents the modular tulip assembly 200 from disassembly from the screwhead 315 while providing polyaxial movement. The modular tulip inserter 100 is then disengaged from the modular tulip assembly 200 and withdrawn W.

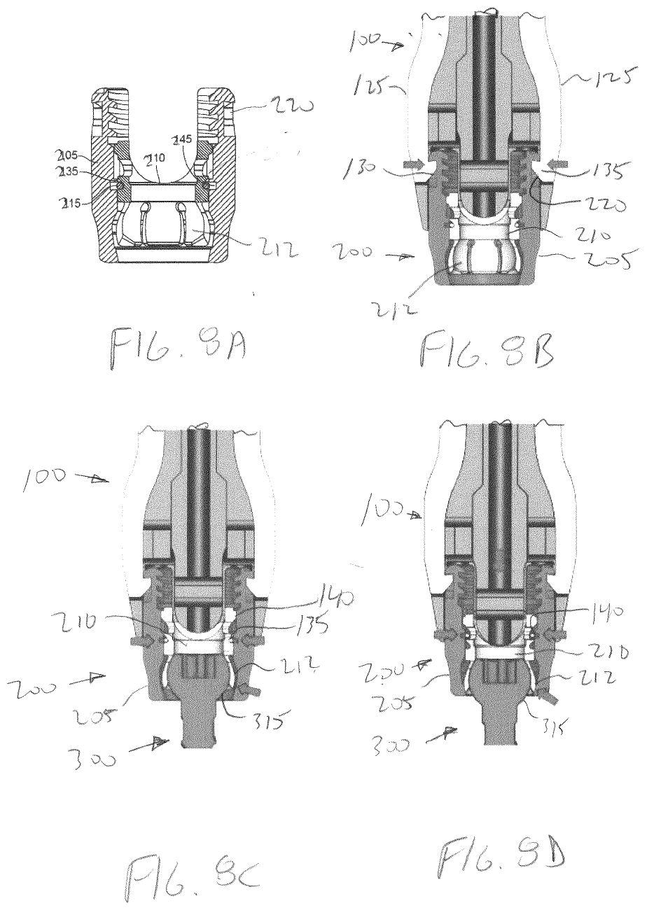

FIGS. 8A-8D are sectional views showing more details of a modular tulip inserter 100 delivering a modular tulip assembly 200 to a pedicle screw 300. During assembly of the modular tulip assembly 200, the modular tulip bushing 210 is inserted into a central opening of the U-shaped tulip body 205 and is translated downward, compressing a flexible wire 215 into a groove slot 145 in the inner wall of the U-shaped tulip body 205 until it reaches the first stage lower cut out 235 in the modular tulip bushing 210, the modular tulip assembly 200 is in an unlocked state. (see FIG. 8A).

The modular tulip inserter 100 includes spring loaded clips 125 with ramped distal ends 130 configured to move the spring-loaded clips 125 apart when they slide on the tulip body 205. The spring-loaded clips 125 further include negative angle engagement tabs 135 and the tulip body includes negative angle pockets 215. The spring-loaded clips 125 slide on the tulip body 205 until the negative angle engagement tabs 135 meet the negative angle pockets 215, then the negative angle engagement tabs 135 go into the negative angle pockets 215, locking the modular tulip assembly 200 on the distal end of the modular tulip inserter 100. The spring-loaded clips 125 maintain the modular tulip assembly 200 during delivery (see FIG. 8B). The modular tulip assembly 200 may be removed from the modular tulip inserter 100 by pressing inward on a proximal end of the spring-loaded clips 125 to disengage the negative angle engagement tabs 135 from negative angle pockets 215 and moving the modular tulip assembly 200 distally.

In the unlocked state, the modular tulip assembly 200 is pushed on the screwhead 315 of the pedicle screw 300 with the modular tulip bushing 210 positioned around the screwhead 315 (FIG. 8C).

Once the screwhead 315 is positioned within the modular tulip assembly 200, the modular tulip inserter 100 pushes the modular tulip bushing 210 downward. During this downward translation, the ramped portion of the lower cut out 135 compresses the flexible wire 115 into the groove slot 145 until it reaches the second stage upper cut out 140, then the flexible wire 115 enters the second stage upper cut out 140, locking the modular tulip bushing 110 in the secured or captured state (see FIG. 8D).

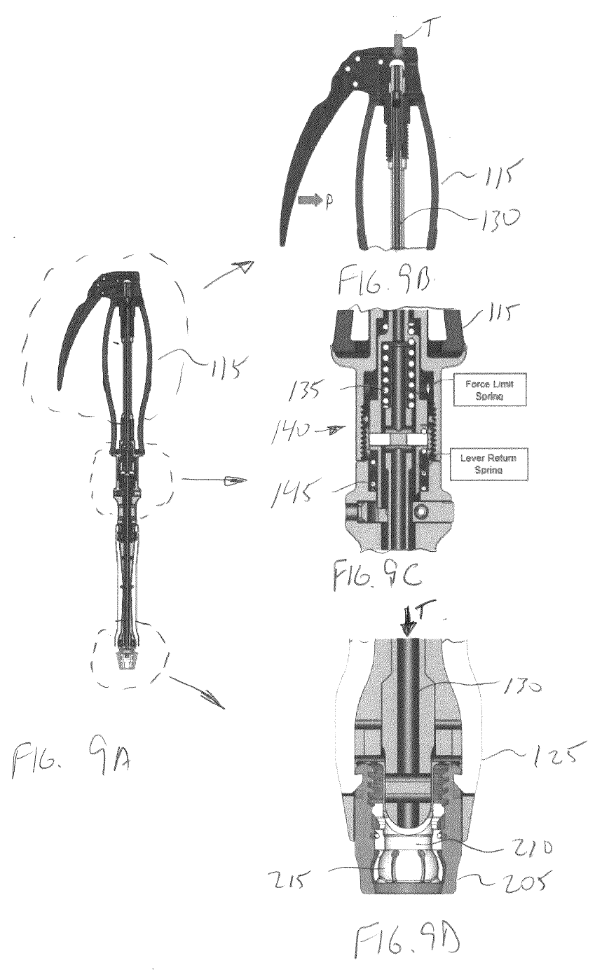

FIGS. 9A-9D are sectional views showing details of a modular tulip inserter 100 with an attached modular tulip assembly 200 for delivery to a screwhead. FIG. 9B shows the proximal end 110 of the modular tulip inserter 100 that includes a handle 115 and a pull lever 120. The handle 115 provides grip for the surgeon to apply a downward force to push the modular tulip assembly 200 onto the screwhead 315 of the screw 300. The handle may be a silicone handle.

The pull lever is connected to an internal shaft 130 via a linkage which converts the pull lever action P to linear translation T of the shaft 130. The proximal end 130 of the internal shaft is coupled via a thread for setting the distal shaft length during assembly. The distal end of the internal shaft 130 contacts the modular tulip bushing 210. When the lever is pulled, the internal shaft 130 translates T to push the modular tulip bushing 210 to the screwhead captured state. The captured state prevents the modular tulip assembly 200 from disassembly from the screwhead 315 while providing polyaxial movement for the screw 300.

A force limiting spring 135 is positioned in an inline coupling 140 attached to the internal shaft 130. The force limiting spring 135 is designed to limit the amount of downward force applied to the modular tulip bushing 210 to avoid taper locking the modular tulip bushing 210 within the U-shaped tulip body 205. which would result in the loss of polyaxial movement of the screw 300. A lever return spring 145 may also be positioned in the inline coupling 140 attached to the internal shaft 130. The lever return spring 145 is designed to compress when the internal shaft 130 is translated when the lever 120 is pulled. When the lever 120 is released, the lever return spring 145 expands and translates the internal shaft 130 upward and returns the lever 120 to the starting position. See FIG. 9C.

FIG. 9D shows the modular tulip assembly 200 coupled to the modular tulip inserter 100, as described above.

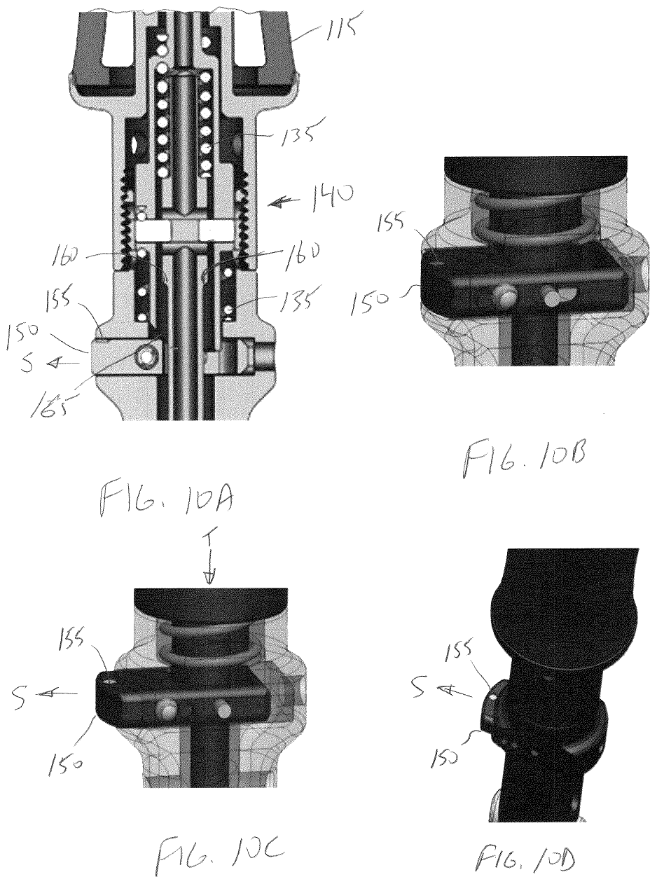

FIGS. 10A-10D show an indicator button 150 with a visual mark 155 positioned in the inline coupling 140 designed to provide visual indication that the modular tulip assembly 200 has been set into the captured state on a screwhead 315. The indicator button 150 is coupled to the internal shaft 130. The internal shaft 130 includes a ramped surface 160 designed to engage a ramped internal cut out 165 of the indicator button 150. During internal shaft 130 translation T, the contact of the ramped surface 160 the ramped internal cut out 165 shifts S the indicator button 150 out of the inline coupling 140 to display visual mark 155 when the internal shaft translates the full distance. The full distance is equivalent to a complete pull of the lever 120. The visual mark 155 provides visual indication that the modular tulip assembly 200 has been set into the captured state. The visual mark 155 may be a white epoxy painted dot.

In use, a modular tulip assembly 200 is selected and attached to the distal end of a modular tulip inserter 100 by way of spring-loaded clips for delivery of the modular tulip to an implanted screw 300. The modular tulip assembly 200 provides grip via a silicone handle 115 to allow downward force to snap the modular tulip assembly 200 onto the screwhead 315 of the screw 300. An audible snap and tactile feedback indicate the modular tulip assembly 200 has completely engaged the screwhead 315 of the screw 300. Next, the surgeon pulls the pull lever 120 all the way in toward the silicone handle 115. The pull lever 120 is connected to an internal shaft 130 via a linkage which converts the pull lever 120 action to linear translation T. The internal shaft 130 contacts the modular tulip bushing 210 and positions it in the captured state on the screwhead 315 The captured state prevents the modular tulip bushing 210 from disassembly from the screwhead 315 while providing polyaxial movement. A force limiting spring 135 limits the amount of downward force onto the modular tulip bushing 210 to avoid taper locking the modular tulip bushing 210 within the U-shaped tulip body 205 which would result in the loss of polyaxial movement of the screw 300.

The modular tulip inserter 100 includes many improvements over prior systems including: i) Controlling the load applied to the modular tulip bushing 210 by way of a force limiting spring 135 to maintain polyaxial movement of the screw 300; ii) Spring loaded clips 125 allowing for quick loading of the modular tulip assembly 200 on the modular tulip inserter 100; iii) Silicone handle 115 with brake lever style lever 120 provides a controlled approach to assembling the modular tulip inserter 100; and iv) Indication button 150 with the visual mark 15 provides visual confirmation of locking the modular tulip bushing 210 within the U-shaped tulip body 205.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A modular tulip inserter designed to attach to a modular tulip assembly comprising:
tulip engagement clips having a distal end configured to attach to a modular tulip body;
an actuation lever on a proximal end; and
an internal shaft having a proximal end connected to the actuation lever and a distal end configured to contact a modular tulip bushing positioned within the modular tulip body;
wherein a pull action of the actuation lever translates the internal shaft distally to apply a downward force to the modular tulip bushing to translate the modular tulip bushing distally in the modular tulip body to a screwhead captured state.

2. The modular tulip inserter of claim 1, further comprising a force limiting spring coupled to the internal shaft designed to limit the amount of downward force applied to the modular tulip bushing.

3. The modular tulip inserter of claim 1, further comprising a lever return spring coupled to the internal shaft configured to:
compress when the actuation lever is pulled, and
expand when the actuation lever is released to return the actuation lever to the starting position.

4. The modular tulip inserter of claim 1, wherein the actuation pull lever is coupled to the internal shaft via a linkage which converts the pull action of the actuation lever to a linear translation of the internal shaft.

5. The modular tulip inserter of claim 1, wherein the internal shaft includes a thread for setting the shaft length.

6. The modular tulip inserter of claim 1, wherein the tulip engagement clips are spring-loaded clips configured to separate when inserted on the modular tulip body until the distal end is coupled to the modular tulip body to lock the modular tulip assembly on the distal end of the modular tulip inserter.

7. The modular tulip inserter of claim 6, wherein pressing inward on a proximal end of the tulip engagement clips disengages the tulip engagement clips from the modular tulip body.

8. The modular tulip inserter of claim 1, further comprising an indicator button having a visual mark designed to provide visual indication when the internal shaft translates distally the full distance.

9. The modular tulip inserter of claim 8, wherein the visual mark is designed to provide visual indication that the modular tulip assembly is in a captured state on the screw.

10. The modular tulip inserter of claim 8, wherein the indicator button includes a ramped internal cut out coupled to a ramped surface on the internal shaft configured to push the indicator button out of the inline coupling during internal shaft translation to display the visual mark when the internal shaft translates the full distance.

11. The modular tulip inserter of claim 1, further comprising a handle on the proximal end configured to provide grip for the surgeon.

12. A modular tulip inserter designed to attach to a modular tulip assembly comprising:
a handle on the proximal end configured to provide grip for a surgeon;
tulip engagement clips having a distal end configured to attach to a modular tulip body;
an actuation lever connected to an internal shaft via a linkage which converts a pull action of the actuation lever to a linear translation of the internal shaft, and a distal end of the internal shaft being configured to contact a modular tulip bushing positioned within the modular tulip body;
wherein the pull action of the actuation lever translates the internal shaft distally to apply a downward force to the modular tulip bushing to translate the modular tulip bushing distally in the modular tulip body to a screwhead captured state; and
a force limiting spring coupled to the internal shaft designed to limit the amount of downward force applied to the modular tulip bushing; and
a lever return spring coupled to the internal shaft configured to:
compress when the actuation lever is pulled, and
expand when the actuation lever is released to return the actuation lever to the starting position.

13. The modular tulip inserter of claim 12, further comprising an indicator button having a visual mark designed to provide visual indication when the internal shaft translates distally a full distance.

14. The modular tulip inserter of claim 13, wherein the indicator button includes a ramped internal cut out coupled to a ramped surface on the internal shaft configured to push the indicator button out during internal shaft translation to display the visual mark when the internal shaft translates the full distance.

15. The modular tulip inserter of claim 12, wherein the tulip engagement clips are spring-loaded clips configured to separate when inserted on the modular tulip body until the distal end is coupled to the modular tulip body to lock the modular tulip assembly on the distal end of the modular tulip inserter.

16. The modular tulip inserter of claim 15, wherein pressing inward on a proximal end of the tulip engagement clips disengages the tulip engagement clips from the modular tulip body.

17. A modular tulip inserter designed to attach to a modular tulip assembly comprising:
a handle on the proximal end configured to provide grip for a surgeon;
tulip engagement spring-loaded clips configured to separate when inserted on a modular tulip body until the distal end is coupled to the modular tulip body to lock the modular tulip assembly on the distal end;
an actuation lever connected to an internal shaft via a linkage which converts a pull action of the actuation lever to a linear translation of the internal shaft, and a distal end of the internal shaft being configured to contact a modular tulip bushing positioned within the modular tulip body;
wherein the pull action of the actuation lever translates the internal shaft distally to apply a downward force to the modular tulip bushing to translate the modular tulip bushing distally in the modular tulip body to a screwhead captured state; and
a force limiting spring coupled to the internal shaft designed to limit the amount of downward force applied to the modular tulip bushing; and
a lever return spring coupled to the internal shaft configured to:
compress when the actuation lever is pulled,
expand when the actuation lever is released to return the actuation lever to the starting position; and
an indicator button having a visual mark designed to provide visual indication when the internal shaft translates distally a full distance.

18. The modular tulip inserter of claim 17, wherein the indicator button includes a ramped internal cut out coupled to a ramped surface on the internal shaft configured to push the indicator button out during internal shaft translation to display the visual mark when the internal shaft translates the full distance.

19. The modular tulip inserter of claim 17, wherein pressing inward on a proximal end of the tulip engagement clips disengages the tulip engagement clips from the modular tulip body.

20. The modular tulip inserter of claim 17, wherein the internal shaft includes a thread for setting the shaft length.
